(19) United States
(12) Patent Application Publication
(10) Pub. No.: US 2010/0210853 A1
(43) Pub. Date: Aug. 19, 2010

(54) PROCESS FOR PREPARATION OF CANDESARTAN CILEXETIL

(75) Inventors: Bandi Parthasaradhi Reddy, Hyderabad (IN); Kura Rathnakar Reddy, Hyderabad (IN); Rapolu Raji Reddy, Hyderabad (IN); Dasari Muralidhara Reddy, Hyderabad (IN); Matta Ramakrishna Reddy, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: HETERO RESEARCH FOUNDATION, Hyderabad, Andhrapradesh (IN)

(21) Appl. No.: 12/678,316

(22) PCT Filed: Jun. 24, 2008
(86) PCT No.: PCT/IN08/00399

§ 371 Date: Mar. 16, 2010

(51) Int. Cl. C07D 257/04

(52) U.S. Cl. 548/253

(57) ABSTRACT

There was provided a process for preparing candesartan cilexetil, the said process comprises hydrogenating a solution of trityl candesartan cilexetil in an alcohol with hydrogen in the presence of a palladium catalyst. Mixture of toluene and methanol was added to 1-(Cyclohexyloxy carbonyloxy)ethyl-2-ethoxy-1-[[2′-(N-triphenylmethyltetrazole-5-yl)biphenyl-4-yl]methyl]benzimidazole-7-carboxylate and hydrogenated at room temperature with hydrogen at atmospheric pressure in the presence of palladium on carbon until the hydrogen uptake was ceased. Filtered over celite bed, washed the bed with a mixture of toluene and methanol, filtrate was collected and concentrated. Co-distilled with acetonitrile, acetonitrile was added, stirred at room temperature, cooled to 0° C. stirred, filtered, washed with chilled acetonitrile and dried to get candesartan cilexetil.

### FIELD OF THE INVENTION

The present invention provides a process for preparation of candesartan cilexetil.

### BACKGROUND OF THE INVENTION

Candesartan cilexetil of formula I:

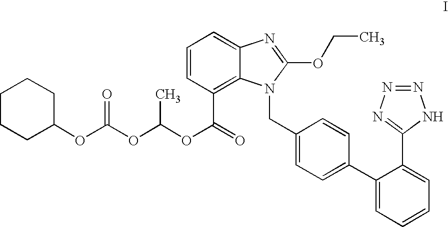

or 2-Ethoxy-1-[[2′-(1H-tetrazol-5-yl)[1,1′-biphenyl]-4-yl]methyl]-1H-benzimidazole-7-carboxylic acid, 1-[[(Cyclohexyloxy)carbonyl]oxy]ethylester is an antihypertensive agent and its therapeutic uses were disclosed in U.S. Pat. No. 5,196,444.

1-[[(Cyclohexyloxy)carbonyl]oxy]ethyl-2-ethoxy-1-[[2′-(N-triphenylmethyltetrazol-5-yl)biphenyl-4-yl]methyl]-1H-benzimidazole-7-carboxylate (herein after referred to as trityl candesartan cilexetil) was a key intermediate in the preparation of candesartan cilexetil. Trityl candesartan cilexetil may be represented by formula II.

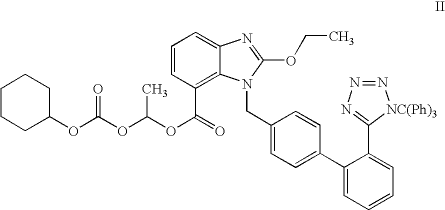

In the prior art, the deprotection of trityl candesartan cilexetil of formula II was carried out by treating trityl candesartan cilexetil with a mineral acid, an organic acid, or a lewis acid catalyst, or by solvolysis in the absence of an acid. WO 2006/015134 A1 discloses the preparation of candesartan by deprotecting silyl protected candesartan by treatment with water or by deprotecting benzyl protected candesartan by hydrogenation of benzyl candesartan suspended in isopropyl alcohol and water.

Candesartan cilexetil was highly sensitive to acids because of the presence of the ester formed by a bulky cilexetil group and because of the presence of the ether group. Therefore, such processes lack reproducibility in terms of yields and purity. Thus, there was a need for a process that was reproducible and commercially viable.

We have developed a process that was reproducible and amicable for scale up from conventionally available trityl candesartan cilexetil. It has been found that the detritylation of protected candesartan cilexetil may be carried out by hydrogenating a solution of protected candesartan cilexetil, in an alcohol with hydrogen in the presence of palladium catalyst. Hydrogenation reaction goes smoothly when protected candesartan cilexetil was in soluble state in an alcohol, even though hydrogenation reaction fails to go when the protected candesartan cilexetil was in a suspended state.

Olmesartan medoxomil chemically 4-(1-Hydroxy-1-methylethyl)-2-propyl-1-[[2′-(1H-tetrazol-5-yl)[1,1′-biphenyl]-4-yl]methyl]-1H-imidazole-5-carboxylic acid (5-methyl-2-oxo-1,3-dioxol-4-yl)methyl ester is an antihypertensive agent and its therapeutic uses were disclosed in U.S. Pat. No. 5,616,599.

4-(1-Hydroxy-1-methylethyl)-2-propyl-1-[[2′-(N-triphenylmethyltetrazol-5-yl)[1,1′-biphenyl]-4-yl]methyl]-1H-imidazole-5-carboxylic acid (5-methyl-2-oxo-1,3-dioxol-4-yl)methyl ester (herein after referred to as trityl olmesartan medoxomil) was a key intermediate in the preparation of olmesartan medoxomil.

We have tried to prepare chemically similar product, olmesartan medoxomil by detritylation of the trityl olmesartan medoxomil by applying the same process used for the preparation of candesartan cilexetil from trityl candesartan cilexetil, but the process has resulted in the formation of olmesartan and not the intended olmesartan medoxomil.

### DETAILED DESCRIPTION OF THE INVENTION

There was provided a process for preparing candesartan cilexetil, the said process comprises hydrogenating a solution of trityl candesartan cilexetil in an alcohol with hydrogen in the presence of a palladium catalyst.

The solution of trityl candesartan cilexetil may be prepared by dissolving trityl candesartan cilexetil in an alcohol. The preferable alcohols were methanol, ethanol and isopropyl alcohol. Any other solvent such as an hydrocarbon solvent carboxylate (100 gm) in ethanol (600 ml) and refluxed for 3 hours 30 minutes then ethanol was removed by distillation and then water (600 ml) and ethyl acetate (600 ml) were added at room temperature, stirred for 30 minutes. Separated the layers, pH of the aqueous layer was adjusted to 4 by using acetic acid at 0° C., stirred for 3 hours, filtered, washed with water (200 ml) and dried to give Candesartan (133 gm, HPLC purity: 97%).

Triethylamine (45 gm) was added to the solution of Candesartan (100 gm) in methylene chloride (500 ml) at 0° C. and then the solution of trityl chloride (85 gm) in methylene chloride (500 ml) was added drop wise at 0° C. for 2 hours and further stirred for 6 hours at room temperature. Water (500 ml) was added to the reaction mass, stirred, separated the layers, and the aqueous layer was extracted with methylene chloride (400 ml). The combined organic layers were washed with water (500 ml), then with 1N hydrochloric acid solution at 4 pH, and with 10% sodium chloride solution (300 ml), dried and then distilled off the organic solvent to obtain a residue. Ethyl acetate (500 ml) and n-Hexane (600 ml) were added to the residue, stirred for 2 hours at room temperature, filtered and washed with mixture of ethyl acetate (50 ml) and n-hexane (50 ml) and then dried to get 2-Ethoxy-1-[[2′-(N-triphenylmethyltetrazole-5-yl)biphenyl)-4-yl]methyl]benzimidazole-7-carboxilic acid (110 gm, High performance liquid chromatography (HPLC) purity: 97%).

Potassium carbonate (60 gm), 1-chloroethylcyclohexyl carbonate (60 gm) and potassium iodide (20 gm) were added to the solution of 2-Ethoxy-1-[[2′-(N-triphenylmethyltetrazole-5-yl)biphenyl)-4-yl]methyl]benzimidazole-7-carboxylic acid (100 gm) in dimethylformamide (500 ml) at room temperature. Raised the temperature to 75° C., stirred for 2 hours, cooled to room temperature and 5% sodium chloride solution (2000 ml) was added. Maintained for 15 minutes, ethyl acetate (400 ml) was added, stirred and separated the layers. Aqueous layer was extracted with ethyl acetate (400 ml), organic layer was taken, washed with 10% sodium chloride solution (400 ml), concentrated, and co-distilled with ethyl acetate (100 ml). Mixture of ethyl acetate (500 ml) and n-hexane (500 ml) were added to the residual mass, stirred for 6 hours at room temperature, cooled to 5° C., stirred for 1 hour, filtered, then washed with mixture of ethyl acetate (50 ml) and n-hexane (200 ml) and dried for 6 hours to obtain 1-(Cyclohexyloxycarbonyloxy)ethyl-2-ethoxy-1-[[2′-(1H-tetrazole-5-yl)biphenyl-4-yl]methyl]benzimidazole-7-carboxylate (110 gm, HPLC Purity: 99%).

Mixture of toluene (1000 ml) and methanol (500 ml) was added to 1-(Cyclohexyloxycarbonyloxy)ethyl-2-ethoxy-1-[[2′-(N-triphenylmethyltetrazole-5-yl)biphenyl-4-yl]methy]benzimidazole-7-carboxylate (100 gm) and hydrogenated at room temperature with hydrogen at 3 atmospheric pressure in the presence of palladium on carbon (10%, 20 gm) until the hydrogen uptake was ceased. Filtered over celite bed, washed the bed with a mixture of toluene (200 ml) and methanol (100 ml), filtrate was collected and concentrated below 45° C. A mixture of acetone (200 ml) and n-hexane (900 ml) was added, stirred at room temperature for 2 hours, cooled to 0° C. and stirred for 4 hours 30 minutes, filtered, washed with a mixture of acetone (20 ml) and n-hexane (180 ml) and dried to get crude candesartan cilexetil (65 gm, HPLC purity: 91%).

Acetone (400 ml) was added to crude candesartan cilexetil, stirred for 30 minutes at reflux, treated with activated carbon and then filtered over celite bed and washed with acetone (50 ml), The filtrate was cooled to room temperature stirred for 30 minutes then added water (500 ml), further stirred at room temperature for 2 hours, filtered and dried the material to yield pure candesartan cilexetil (56 gm, HPLC purity: 99%).

### Example 2

Mixture of toluene (600 ml) and ethanol (300 ml) was added to 1-(Cyclohexyloxycarbonyloxy)ethyl-2-ethoxy-1-[[2′-(N-triphenylmethyltetrazole-5-yl)biphenyl-4-yl]methyl]benzimidazole-7-carboxylate (60 gm) and hydrogenated at room temperature with hydrogen at 1 atmospheric pressure in the presence of palladium on carbon (10%, 12 gm) until the hydrogen uptake was ceased. Filtered over celite bed, washed with a mixture of toluene (100 ml) and ethanol (50 ml), filtrate was collected and concentrated below 45° C. A mixture of acetone (100 ml) and n-hexane (450 ml) was added, stirred at room temperature for 2 hours, cooled to 0° C. and stirred for 4 hours 30 minutes, filtered, washed with a mixture of acetone (10 ml) and n-hexane (90 ml) and dried to get crude candesartan cilexetil (39 gm, HPLC purity: 92%).

Acetone (200 ml) was added to crude candesartan cilexetil, stirred for 30 minutes at reflux, treated with activated carbon and then filtered over celite bed and washed with acetone (30 ml), The filtrate was cooled to room temperature stirred for 30 minutes then added water (300 ml), further stirred at room temperature for 2 hours, filtered, crystallized from ethanol and dried the material to yield pure candesartan cilexetil (32 gm, HPLC purity: 99.2%).

### Example 3

Mixture of toluene (500 ml) and isopropanol (250 ml) was added to 1-(Cyclohexyloxycarbonyloxy)ethyl-2-ethoxy-1-[[2′-(N-triphenylmethyltetrazole-5-yl)biphenyl-4-yl]methyl]benzimidazole-7-carboxylate (50 gm) hydrogenated at room temperature with hydrogen at 2 atmospheric pressure in the presence of palladium on carbon (10%, 10 gm) until the hydrogen uptake was ceased. Filtered over celite bed, washed with a mixture of toluene (50 ml) and isopropanol (50 ml), filtrate was collected and concentrated below 45° C. Co-distilled with n-hexane (50 ml), n-hexane (500 ml) was added, stirred at room temperature for 30 minutes, filtered. Acetonitrile (250 ml) was added, stirred at room temperature for 30 minutes, cooled to 0° C. and stirred for 2 hours 30 minutes, filtered, washed with chilled acetonitrile (50 ml), and dried to get crude candesartan cilexetil (32 gm, HPLC purity: 90%).

Acetonitrile (200 ml) was added to crude candesartan cilexetil, stirred for 20 minutes, refluxed, treated with activated carbon and then filtered, The filtrate was cooled to room temperature, stirred for 30 minutes, further stirred at 0° C. for 2 hours, filtered, washed with chilled acetonitrile (30 ml). The above purification in acetonitrile was repeated and dried the material to yield pure candesartan cilexetil (24 gm, HPLC purity: 99.4%).

### Example 4

Mixture of ethyl acetate (400 ml) and methanol (200 ml) was added to 1-(Cyclohexyloxycarbonyloxy)ethyl-2-ethoxy-1-[[2′-(N-triphenylmethyltetrazole-5-yl)biphenyl-4-yl]methyl]benzimidazole-7-carboxylate (40 gm) and hydrogenated at room temperature with hydrogen at 2 atmospheric pressure in the presence of palladium on carbon (10%, 8 gm) until the hydrogen uptake was ceased. Filtered over celite bed, washed with a mixture of ethyl acetate (40 ml) and methanol (40 ml), filtrate was collected and concentrated below 45° C. Co-distilled with acetonitrile (40 ml), acetonitrile (200 ml) was added, stirred at room temperature for 30 minutes, cooled to 0° C. and stirred for 2 hours 30 minutes, filtered the solid, washed with chilled acetonitrile (40 ml) and dried to get crude candesartan cilexetil (25 gm, HPLC purity: 92%).

Acetonitrile (150 ml) was added to crude candesartan cilexetil, stirred for 20 minutes, refluxed, treated with activated carbon and then filtered. The filtrate was cooled to room temperature stirred for 30 minutes, further stirred at 0° C. for 2 hours, filtered, filtered, washed with chilled acetonitrile (25 ml). The above purification in acetonitrile was repeated and dried the material to yield of pure candesartan cilexetil (21.8 gm, HPLC purity: 99.6%).

### Example 5

Mixture of toluene (250 ml) and methanol (125 ml) was added to 1-(Cyclohexyloxycarbonyloxy)ethyl-2-ethoxy-1-[[2′-(N-triphenylmethyltetrazole-5-yl)biphenyl-4-yl]methyl]benzimidazole-7-carboxylate (25 gm) and hydrogenated at room temperature with hydrogen at 3 atmospheric pressure in the presence of palladium on barium sulphate (2.5 gm) until the hydrogen uptake was ceased. Filtered over celite bed, washed with a mixture of toluene (50 ml) and methanol (25 ml), filtrate was collected and concentrated below 45° C. Co-distilled with acetonitrile (40 ml), acetonitrile (150 ml) was added, stirred at room temperature for 30 minutes, cooled to 0° C. and stirred for 2 hours 30 minutes, filtered the solid, washed with chilled acetonitrile (25 ml) and dried to get crude candesartan cilexetil (16 gm, HPLC purity: 90%).

Acetonitrile (90 ml) was added to crude candesartan cilexetil, stirred for 20 minutes, refluxed, treated with activated carbon and then filtered. The filtrate was cooled to room temperature stirred for 30 minutes, further stirred at 0° C. for 2 hours, filtered, washed with chilled acetonitrile (15 ml). The above purification in acetonitrile was repeated and dried the material to yield pure candesartan cilexetil (12 gm, HPLC purity: 99.4%).

### Example 6

Mixture of toluene (500 ml) and methanol (250 ml) was added to 1-(Cyclohexyloxycarbonyloxy)ethyl-2-ethoxy-1-[[2′-(N-triphenylmethyltetrazole-5-yl)biphenyl-4-yl]methyl]benzimidazole-7-carboxylate (50 gm) hydrogenated at room temperature with hydrogen at 1 atmospheric pressure in the presence of palladium on carbon (10%, 10 gm) until the hydrogen uptake was ceased. Filtered over celite bed, washed with a mixture of toluene (100 ml) and methanol (50 ml), filtrate was collected and concentrated below 45° C. Co-distilled with cyclohexane (50 ml), cyclohexane (500 ml) was added, stirred at room temperature for 30 minutes, filtered. Acetonitrile (250 ml) was added, slurred at room temperature for 30 minutes, Cooled to 0° C. and stirred for 2 hours 30 minutes, filtered, washed with chilled acetonitrile (50 ml), and dried to get crude candesartan cilexetil (32 gm, HPLC purity: 92%).

Acetonitrile (180 ml) was added to crude candesartan cilexetil, stirred for 20 minutes, refluxed, treated with activated carbon and then filtered, The filtrate was cooled to room temperature, stirred for 30 minutes, further stirred at 0° C. for 2 hours, filtered, washed with chilled acetonitrile (25 ml). The above purification in acetonitrile was repeated and dried the material to yield pure candesartan cilexetil (26 gm, HPLC purity: 99.5%).

### Example 7

Mixture of toluene (1000 ml) and methanol (500 ml) was added to 1-(Cyclohexyloxycarbonyloxy)ethyl-2-ethoxy-1-[[2′-(N-triphenylmethyltetrazole-5-yl)biphenyl-4-yl]methyl]benzimidazole-7-carboxylate (100 gm) and hydrogenated at room temperature with hydrogen at 3 atmospheric pressure in the presence of palladium on carbon (10%, 20 gm) until the hydrogen uptake was ceased. Filtered over celite bed, washed the bed with a mixture of toluene (200 ml) and methanol (100 ml), filtrate was collected and concentrated below 45° C. Co-distilled with acetonitrile (100 ml), acetonitrile (500 ml) was added, stirred at room temperature for 30 minutes, cooled to 0° C. and stirred for 2 hours 30 minutes, filtered, washed with chilled acetonitrile (100 ml) and dried to get crude candesartan cilexetil (66 gm, HPLC purity: 91%).

Acetonitrile (300 ml) was added to crude candesartan cilexetil, stirred for 30 minutes at reflux, treated with activated carbon and then filtered over celite bed and washed with acetonitrile (50 ml), The filtrate was cooled to room temperature stirred for 30 minutes, further stirred at 0° C. for 2 hours, filtered, washed with chilled acetonitrile (50 ml). The above purification in acetonitrile was repeated and dried the material to yield pure candesartan cilexetil (51 gm, HPLC purity: 99.5%).

1) A process for preparing candesartan cilexetil, the said process comprises hydrogenating a solution of trityl candesartan cilexetil in an alcohol in a substantially anhydrous condition with hydrogen in the presence of a palladium catalyst.

2) The process according to claim 1, wherein palladium catalyst used is supported on carbon, calcium carbonate, barium sulfate or alumina.

3) The process according to claim 2, wherein the catalyst is palladium supported on carbon.

4) The process according to claim 1, wherein palladium catalyst used is palladium oxide.

5) The process according to claim 1, wherein alcohol is methanol, ethanol or isopropyl alcohol.

6) The process according to claim 1, wherein alcohol is mixed with a hydrocarbon solvent.

7) The process according to claim 1, wherein hydrocarbon solvent is toluene.

8) The process according to claim 1, wherein hydrogenation is carried out at reflux temperature of the solvent medium or below.

9) The process according to claim 1, wherein hydrogenation is carried out under the hydrogen pressure of 1 to 10 atmospheres.

10) The process according to claim 9, wherein hydrogenation is carried out under the hydrogen pressure of 1 to 5 atmospheres.
